(19) United States
(12) Patent Application Publication
(10) Pub. No.: US 2015/0329811 A1
(43) Pub. Date: Nov. 19, 2015

(54) Method of Using A Process Vessel

(71) Applicant: Evergreen Gas Limited, Ludlow, Shropshire (GB)

(72) Inventor: Michael James Chesshire, Ludlow (GB)

(21) Appl. No.: 14/379,257

(22) PCT Filed: Feb. 15, 2013
(86) PCT No.: PCT/GB2013/050359

§ 371 Date: Aug. 15, 2014

(30) Foreign Application Priority Data
Feb. 15, 2012 (GB) 1202612.6
Feb. 15, 2012 (GB) 1202615.9
Feb. 29, 2012 (GB) 1203537.4
Feb. 29, 2012 (GB) 1203548.1

(51) Int. Cl. C12M 1/107; C12M 1/12

(52) U.S. Cl. CPC C12M 21/04; C12M 23/36; C12M 25/06

(57) ABSTRACT

According to the invention there is provided a process vessel for use in a process in which a liquid charge produces a gaseous product including:
a receptacle for receiving the liquid charge, the receptacle having an open upper end; and
a plurality of closure portions for closing said open upper end, in which each closure portion is configured to provide a headspace in which the gaseous product can collect, and at least one closure portion is operable separately to other closure portions to permit access to the interior of the receptacle;
wherein the process vessel is configured so that the liquid charge extends continuously within the process vessel, and when liquid charge is present in the process chamber at or above a predetermined level, the gaseous products in each headspace are isolated from each other, at least in part by the liquid charge.

This invention relates to a method of using a process vessel in a process in which a liquid charge produces a gaseous product, with particular, but by no means exclusive, reference to anaerobic digestion. The invention relates also to associated process vessels and kits of parts.

Conventional anaerobic digesters are not readily accessible for internal inspection, maintenance or cleaning. In particular, anaerobic digesters comprise a receptacle such as a tank body and a separate lid which are sealed together to form a chamber in which the anaerobic digestion takes place. It is common to use a sealant such as a Mastic® to hermetically seal the lid to the tank body. It is difficult to subsequently remove the lid from the tank, and this can only be done by permanently breaking the seal. In another prior art arrangement, it is known to use a water seal in conjunction with a heavy, floating lid. It is at best very difficult to remove the heavy lid in order to gain access to the interior of the anaerobic digester. In all of these cases, biogas produced by the anaerobic digestion process is lost when the lid is removed. Normally the liquid contents of the digester are removed prior to access into the interior of the anaerobic digester. Therefore, operation the digester must cease whilst the interior is accessed, which is economically undesirable.

A major problem with anaerobic digestion is the removal of grit which can build up inside an anaerobic digester. This is particularly challenging for biodegradable feedstocks obtained from dairy farms, where the cows are bedded on sand which is often considered to be the optimum bedding material. The associated biodegradable feedstock fed into an anaerobic digester will certainly contain grit or sand which subsequently requires removal. It will be apparent from the foregoing discussion that this is difficult, impractical, or even impossible with conventional prior art anaerobic digesters. In one known anaerobic digester arrangement, a mechanical scraper is provided for removing grit. However, this adds to production costs and is vulnerable to mechanical failure. In the event of a mechanical failure, the digester must be drained of its liquid contents in order to access the scraper, which is undesirable.

The present invention, in at least some of its embodiments, addresses the above described problems.

For the avoidance of doubt, the term ‘liquid’ as used herein includes reference to slurries.

According to a first aspect of the invention there is provided a process vessel for use in a process in which a liquid charge produces a gaseous product including:

a receptacle for receiving the liquid charge, the receptacle having an open upper end; and

a plurality of closure portions for closing said open upper end, in which each closure portion is configured to provide a headspace in which the gaseous product can collect, and at least one closure portion is operable separately to other closure portions to permit access to the interior of the receptacle;

wherein the process vessel is configured so that the liquid charge extends continuously within the process vessel, and when liquid charge is present in the process chamber at or above a predetermined level, the gaseous products in each headspace are isolated from each other, at least in part by the liquid charge.

In this way, the above described problems may be overcome. A further advantage is that it is possible to replace a single closure portion which has, for example, suffered damage. This can be done without replacing other closure portions, and the replacement of one closure portion can be performed with minimal or even no disturbance of other closure portions.

Generally, the closure portions are non-floating.

Typically, said at least one closure portion is removable from the process vessel separately to other closure portions to permit access to the interior of the receptacle.

Preferably, each closure portion is configured to receive, in use, a portion of the liquid charge so that the gaseous products in each headspace are isolated from each other by the liquid charge present in each closure portion.

Alternatively, the receptacle may be provided with one or more divider structures which are located at the open upper end so as to divide at least one closure portion from another closure portion. In this instance, the predetermined level of the liquid charge which is required to isolate the gaseous products in each headspace from each other may be lower. In these embodiments, the combination of the liquid charge and the divider structures acts to isolate the gaseous products in each headspace from each other. The term ‘open upper end’ is understood to encompass arrangements in which the upper region of the receptacle comprises dividers, apertures or other upper internal structures, ie, an upper region which is not entirely open, as well as arrangements which are entirely open.

It is understood that in the invention the receptacle is not in the form of separate compartments or chambers. Instead, in use the liquid charge extends continuously within the process vessel. Any divider structures at the open upper end would not extend to the lower portion of the receptacle to form separate compartments or chambers.

Preferably, the process vessel is in the form of anaerobic digester. However, the invention extends to other process vessels, such as continuous stirred tank reactors (CSTR), micro-digesters, vessels for brewing, and vessels for various chemical, chemical engineering and biological processes.

Advantageously, the process vessel includes three or more closure portions. In the case of an anaerobic digester, it is possible to provide nine to 11 closure portions or more.

Advantageously, at least some of the closure portions consist essentially of a polymeric material or a composite material. Examples of suitable materials are glass-reinforced plastic (GRP) or a polyolefin, such as polypropylene.

For anaerobic digesters, the closure portions (or at least those areas of the closure portions which are in contact with biogas during normal operation) should be formed from a corrosion-resistant material, in particular a material which is resistant to corrosion by hydrogen sulphide. Polymeric materials, composite materials, stainless steel or a membrane structure might be utilised.

In some embodiments, the process vessel includes a plurality of closure portions in combination with a fixed roof structure. For anaerobic digesters, it is advantageous to provide one or more closure portions located over areas in which grit or sand is expected to accumulate. These closure portions are provided in combination with a fixed roof structure which covers the remainder of the open end of the receptacle. Typically, three or four closure portions are provided. The fixed roof structure may be formed from a rigid material such as concrete.

Preferably, at least one of the closure portions is tied to the receptacle by a tie arrangement which prevents or limits movement of the closure portion with respect to the receptacle.

The present invention provides an improved means of sealing the receptacle and closure portion of a process vessel. In the field of anaerobic digesters, it is typical in the prior art for the interface between the closure portion and the receptacle to be in contact with the biogas during actual use. The present inventors have recognised that it is instead highly advantageous for the interface to be contacted, in use, by the liquid charge. The present inventors have exploited this realisation by providing a process vessel which is configured to use the liquid charge to form a liquid seal around the interface. This obviates any need to provide a hermetic seal, such as by using a sealant. Further advantages are discussed in more detail below.

The use of a tie arrangement in conjunction with the liquid seal enables the closure portions to be fabricated from a relatively lightweight and/or inexpensive material. In the case of anaerobic digesters, the closure portions are generally fabricated from a suitable corrosion resistant material, typically a material which is resistant to corrosion by hydrogen sulphide.

Generally, the tie arrangement is releasable so as to allow the closure portion to be untied from the receptacle and subsequently retied to the receptacle. This is highly advantageous, since it permits convenient and repeated access to the interior of the receptacle for, e.g., cleaning or maintenance purposes.

Advantageously, the tie arrangement is a mechanical arrangement. The mechanical arrangement may consist of a plurality of bolts which fix the closure portion to the receptacle, although a great many mechanical arrangements might be utilised within the scope of the invention. Such arrangements would readily suggest themselves to the skilled person. Other tie arrangements might be envisaged, such as an electromechanical arrangement or an electromagnetic arrangement.

Generally, the tie arrangements of the invention fall within two broad categories. The first category is a fixing arrangement which essentially prevents movement of the closure portion of the receptacle. The second category is a movement limiting arrangement which limits the movement of the closure portion with respect to the receptacle, for example by providing a flexible tie arrangement or by providing a limit stop on the movement of the closure portion. Mechanical arrangements are particularly preferred owing to their simplicity, economic viability and ease of use.

Conveniently, the receptacle includes an internal flange portion which receives at least some of the closure portions. Advantageously, at least some of the closure portions include a rim which is received by the internal flange portion. At least some of the closure portions may include a rim which is received by a rim of an adjacent closure portion so that the closure portions extend between opposite walls of the receptacle without any additional elements therebetween.

Advantageously, each of the closure portions have an associated gas outlet pipe which extends through a wall of the receptacle. Typically, each gas outlet pipe has valve means which can isolate the gas outlet pipe from its associated closure portion.

The process vessel may further include one or more walls disposed on the floor of the receptacle. The wall or walls may be used to prevent or inhibit the movement of grit or sand across the floor of the receptacle. The wall or walls are typically quite low. A representative but non-limiting wall height is 500 mm. The wall or walls are typically disposed in areas in which grit or sand is expected to accumulate. Generally, the wall or walls are each disposed underneath a closure portion which is operable separately to other closure portions to permit access to the interior of the receptacle. In this way, the grit or sand accumulating in the process vessel is more easily removed through the removal of a limited number of closure portions.

Plug flow may be achieved using process vessels of the invention, particularly in connection with anaerobic digesters.

According to a second aspect of the invention there is provided a kit of parts for assembling a process vessel according to the first aspect of the invention.

According to a third aspect of the invention there is provided a method of using a process vessel in a process in which a liquid charge produces a gaseous product, the method including the steps of:

providing a process vessel which includes a receptacle having an open upper end, and a plurality of closure portions for closing said open upper end, in which the process vessel is configured so that the liquid charge extends continuously within the process vessel; each closure portion is configured to provide a headspace in which the gaseous product can collect, and at least one closure portion is operable separately to other closure portions to permit access to the interior of the receptacle;

filling the process vessel with liquid charge to a level; and

collecting the gaseous product in the headspaces provided by the closure portion;

wherein liquid charge is present in the process chamber at or above a predetermined level so that the liquid charge, at least in part, isolates the gaseous products in each headspace from each other.

Typically, the closure portion is operated by removing it from the process vessel to permit access to the receptacle interior.

Advantageously, the process vessel is filled so that each closure portion receives liquid charge, and the gaseous products in each headspace are isolated from each other by liquid charge present in each closure portion.

Advantageously, the method includes the further step of operating one or more of the closure portions separately to other closure portions to permit access to the interior of the receptacle. In this way, access can be gained to part of the interior of the process vessel whilst the remainder of the process vessel is essentially undisturbed. In particular, process gas present within the other closure portions (which have not been removed) is not lost by opening the process vessel. Also, it is not necessary to drain the liquid charge from the receptacle prior to access to the interior of the receptacle. This allows access to the interior of the process vessel for a variety of purposes. In one preferred embodiment, the process vessel is an anaerobic digester, and access is gained for the purpose of removing sand or grit from the anaerobic digester. This can be achieved without draining the liquid charge from the receptacle. In these preferred embodiments, the anaerobic digester may include an inlet for introducing liquid charge or a semi-solid charge into the process vessel. In these embodiments, the closure portion which is overhead the area of the receptacle in which liquid charge is first introduced into the process vessel may be removed separately to the other closure portions. This is because it is in this area of the receptacle that sand or grit principally accumulates.

Whilst the invention has been described above, it extends to any inventive combination of the features set out above, or in the following description, drawings or claims.

Embodiment of process vessels in accordance with the invention will now be described with reference to the accompanying drawings, in which:—

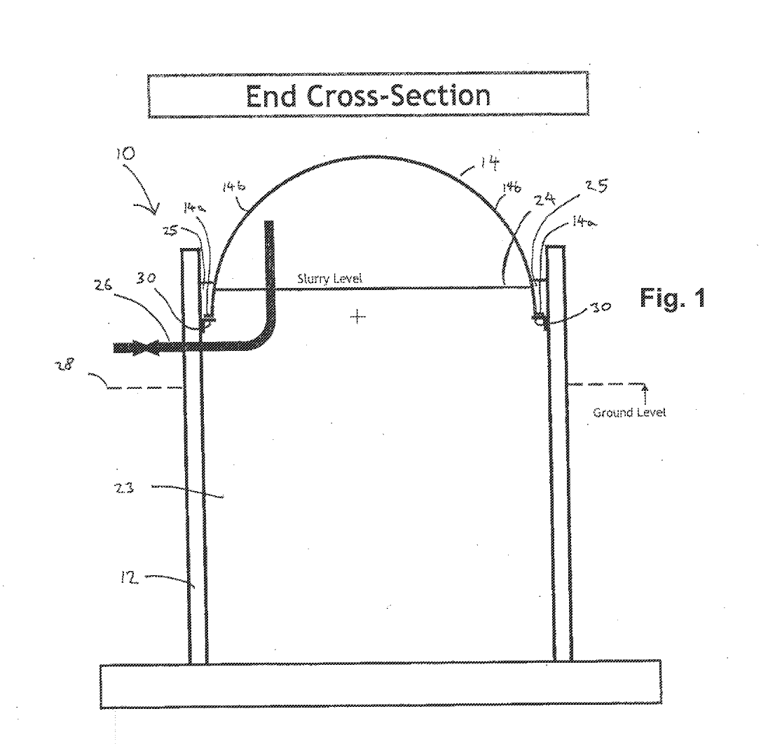

FIG. 1 is an end cross section of a process vessel of the invention;

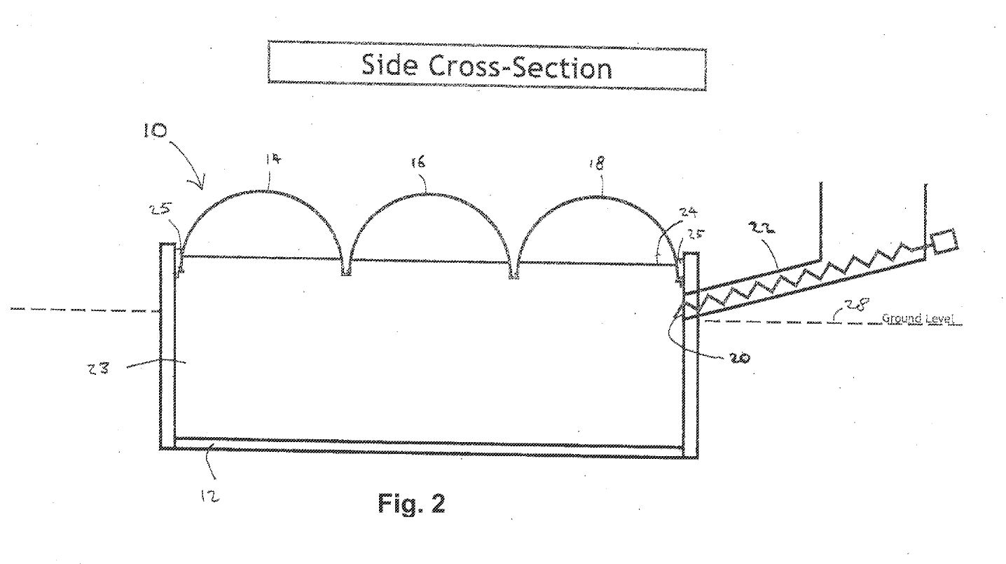

FIG. 2 is a side cross section of a process vessel of the invention; and

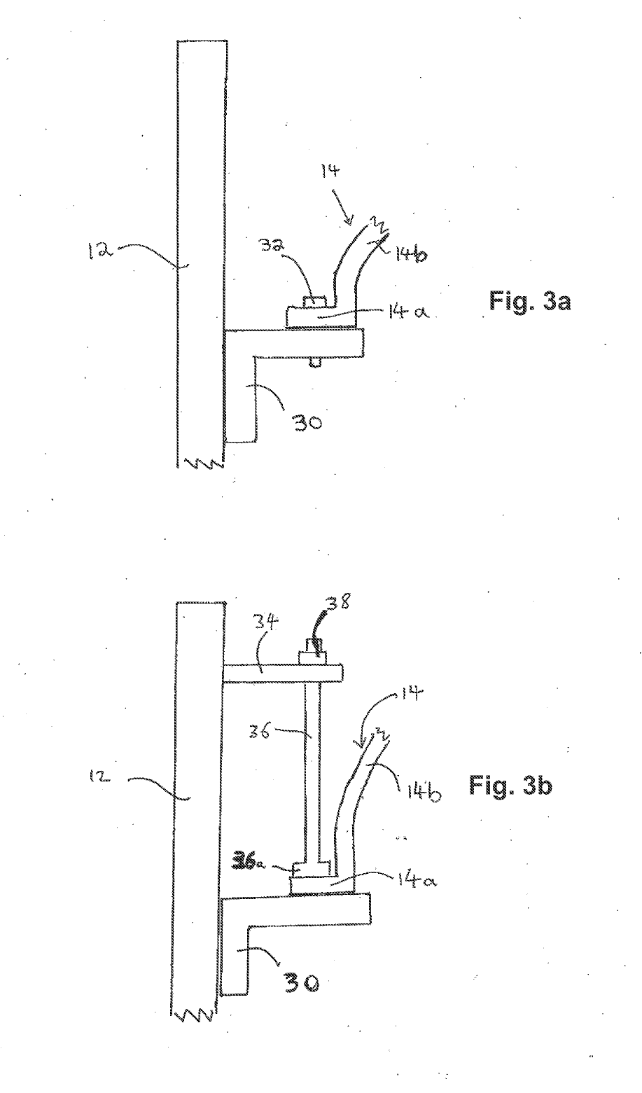

FIG. 3 shows (a) a first and (b) a second embodiment of a fixing arrangement.

The Figures show an anaerobic digester process vessel, depicted generally at 10, of the invention. The process vessel comprises a receptacle 12 and a roof structure in the form of three closure portions 14, 16, 18. The provision of the three closure portions as shown in the Figures is purely for example only. In particular, it is often desirable to utilise a greater number of closure portions, such as five or more. The receptacle 12 has an inlet 20 for a biodegradable feedstock. The biodegradable feedstock may be introduced to the anaerobic digester as a slurry. Alternatively, it is possible to introduce the biodegradable feedstock as a semi-solid. FIG. 2 shows the latter example, where an auger feeder 22 conveys a semi-solid to the inlet 20. Although in this embodiment the biodegradable feedstock is initially in the form of a semi-solid, the anaerobic digestion process subsequently converts the feedstock to a slurry as the solid materials are biodegraded whilst water present in the semi-solid is retained. This results in a slurry 23 which fills the anaerobic digester 10 up to a level indicated by 24. Alternatively, the feedstock may be introduced to the receptacle in the form of a slurry. It should be noted that the level 24 is such that the slurry is present in each of the closure portions 14, 16, 18. The remainder of each closure portion 14, 16, 18 acts as a headspace in which biogas generated by the anaerobic digestion process collects. Each of the closure portions 14, 16, 18 has an associated gas outlet pipe which extends through the wall of the receptacle 12 and enables the generated biogas to be removed from the anaerobic digester 10. Each gas outlet pipe has a valve which can isolate the gas outlet pipe from its associated closure portion. FIG. 1 shows the gas outlet pipe 26 is associated with the closure portion 14.

The receptacle 12 is deliberately filled with an amount of the feedstock sufficient to result in a slurry which fills the anaerobic digester 10 to a level at which the slurry extends into the closure portions 14, 16, 18. The Figures show an example of this where the level 24 is such that slurry 23 is present in each of the closure portions 14, 16, 18. This has the effect of isolating the headspaces of the closure portions 14, 16, 18 from one another. This has the advantage that it is possible to remove one of the closure portions without disturbing the biogas stored by the other closure portions. This means that access to the anaerobic digester 10 is possible without halting operation of the digester. For example, it is possible to remove the closure portion 18 whilst leaving the other closure portions 14, 16 undisturbed. It should be noted that the valve of the associated gas outlet pipe is closed prior to removal of a closure portion. The facility to access a desired portion of the anaerobic digester 10 is advantageous, particularly in connection with the removal of grit which can build up inside an anaerobic digester. It is likely that the majority of the build up of grit will occur in the region close to the inlet 20, and therefore it is anticipated that access to the interior of the anaerobic digester 10 located below the closure portion 18 will allow the majority of the accumulated grit to be removed. It is extremely convenient that the amount of grit present can be assessed very easily once access has been gained to the interior of the digester, for example by positioning a stick in the receptacle.

Advantageously, the receptacle 12 is partially buried in the ground. In the Figures, the ground level is indicated by 28. This allows convenient removal of grit using an excavator to dredge the receptacle 12. It is advantageous that all vulnerable equipment is installed on one side of the receptacle, and thus the gas outlet pipes such as the pipe 26 are all installed on one side of the receptacle 12. A suitable device such as an excavator can then be operated from the side opposite to the side on which the vulnerable equipment is located. If it is judged that sufficient grit is accumulated elsewhere in the anaerobic digester 10 to require further dredging, then it is possible to remove further closure portions. In the embodiment shown in the Figures, if removal of closure portion 18 is not sufficient to enable the grit to be removed, then the closure portion 16 could be removed to enable greater access to the receptacle 12. This would still permit anaerobic digestion to take place using the closure portion 14 to collect biogas. In other embodiments having a greater number of closure portions, removal of two closure portions would result in less disruption to the anaerobic digestion process.

The receptacle 12 is provided with an internal flange 30. The internal flange 30 is disposed around the entire perimeter of the internal walls of the receptacle 12. Conveniently, the internal flange 30 can be provided as a 90° bracket fabricated from a suitable material such as a metal, although other embodiments would readily suggest themselves to the skilled reader. However, it is convenient that standard fixtures, such as a 50×50 mild steel angle, can be used for this purpose. The flange 30 is fixed to the receptacle 12 by way of a gas-tight seal. This can be achieved using a sealant such as a Mastic®. In this way, bubbles of biogas travelling upwards along an internal wall of the receptacle 12 cannot escape from the digester at the flange/receptacle wall interface. The closure portions 14, 16, 18 are each in the shape of a dome. As shown at FIG. 1, the closure portion 14 has a rim portion 14a and a hemispherical roof structure 14b. In this embodiment the rim structure 14a is essentially square in plan view, and the other closure portions 16, 18 are of identical design and construction. The rim structure 14a abuts the flange 30 on three sides to form an interface between the receptacle 12 and the closure portion 14. It can be seen from FIG. 2 that on a fourth side, the rim structure 14a abuts a similar rim structure of the closure portion 16. More specifically, the rim structure 14a is located beneath the rim structure of the closure portion 16, On the opposite side of the closure portion 16, the rim structure of closure portion 18 is located above the rim structure of closure portion 16. It will be appreciated that the rim structure of the closure portion 16 abuts the flange 30 on two sides to form an interface between the receptacle 12 and the closure portion 16. The hemispherical roof structure provides excellent structural strength in resisting gas pressure. However, the closure portions can be of any suitable size and dimensions. A particularly preferred shape is a generally half-obround shape, ie, one half of an obround which has been divided by a plane passing through its axis of symmetry. Other possible shapes for the closure portions include a generally Quonset® shape and a generally half-ellipsoid shape. A Quonset® shape is understood to refer to a shape consisting of one-half of a right circular cylinder which has been divided by a plane passing through its axis of symmetry.

The closure portion 14 is fixed to the receptacle 12 by a fixing arrangement. For ease of presentation, the fixing arrangement is not shown in FIGS. 1 and 2. FIGS. 3a and 3b show two embodiments of a fixing arrangement. FIGS. 3a and 3b share numerous features with FIGS. 1 and 2, and identical reference numerals are used to denote such shared features. FIG. 3a shows a simple arrangement in which bolts 32 are used to secure the rim structure 14a to the flange 30 using threaded apertures (not shown) in the flange 30. FIG. 3b shows a clamping arrangement comprising a horizontal bar which has a vertical internal aperture (not shown) which receives a clamp 36. The clamp 36 is positioned vertically, and has an enlarged clamping head 36a which engages the rim structure 14a. The clamp 36 can be retained in the horizontal bar 34 by any convenient means. For example, a nut 38 may be used in conjunction with a clamp 36 which is flooded at the end distal from the clamping head 36a. The horizontal bar 34 may be retained in a socket (not shown) disposed in the wall of the receptacle 12. The fixing arrangement is such as to allow a liquid seal to be formed between the closure portions and the exterior of the anaerobic digester. More specifically, the fixing arrangement is such as to permit egress of the slurry to form an exterior reservoir 25. The external reservoir 25 is retained by a suitable reservoir boundary structure, which most conveniently is simply provided by the uppermost portions of the upstanding walls of the receptacle 12. It will be noted that the level of the external reservoir 25 is slightly higher than the level 24 of the slurry 23. This is due to the gas pressure exerted by the biogas in the headspaces, which typically is the order of 50 to 100 mm water gauge.

Therefore, the present invention does not require the provision of a gas impermeable seal, and it is not necessary, or even desirable, to use a sealant composition. It will be apparent to the skilled reader that there are a great many ways in which the liquid seal can be achieved. For example, spacers might be provided between the rim structure 14a and the flange 30. Alternatively, the fixing arrangement (which might utilise bolts or clamps) might be installed to provide a certain amount of “give”, for example by not fully tightening a bolt or spacing a clamp head from the rim structure 14a. Alternatively still, a tie might be used which restricts the range of motion of the closure portion with respect to the flange 30, but otherwise provides no fixing effect.

The scheme of ensuring that the level 24 of the slurry extends into the closure portions has further advantages. A consequence of this mode of operation is that the receptacle 12 is not exposed to corrosive biogas. Accordingly, it is possible to manufacture the receptacle 12 from a convenient but non-corrosive resistant material such as concrete. The approach of securing the closure portions to the receptacle using a fixing arrangement has the advantage that the closure portions do not have to be manufactured as heavy items. The closure portions can instead be manufactured as relatively light weight structures from suitable polymeric or composite materials such as glass-reinforced plastic (GRP) or polypropylene, which are corrosion resistant (in particular resistant to corrosion from hydrogen sulfide which is present in biogas).

Typically, the anaerobic digesters of the invention will be utilised in combination with features such as a discharge arrangement for discharging digestate from the receptacle, a feeder storage facility which can transfer feedstock to the auger feeder, and a suitable gas holder for retaining the generated biogas. It will be apparent that the embodiment discussed herein is an example only, and that many variations are possibly utilised in the principles of the invention. For example, the closure portions are typically operable separately to other closure portions to permit access to the interior of the receptacle by way of removing the closure portion from the receptacle. However, in principle other ways of achieving selective access to the interior of the receptacle might be contemplated, such as removal of a section of the closure portion, or providing a slideable or hinged panel which can be moved to permit access whilst the closure portion remains in connection to the receptacle. Whilst dome shaped closure portions can be convenient, owing to their structural strength, many other structures are possible. Similarly, the materials used and the number of closure portions may be varied. Although the example provided above relates to an anaerobic digester, it is possible that the invention may be applied in other areas, such as in brewing.

1. A process vessel for use in a process in which a liquid charge produces a gaseous product including:
a receptacle for receiving the liquid charge, the receptacle having an open upper end; and
a plurality of closure portions for closing said open upper end, in which each closure portion is configured to provide a headspace in which the gaseous product can collect, and at least one closure portion is operable separately to other closure portions to permit access to the interior of the receptacle;
wherein the process vessel is configured so that the liquid charge extends continuously within the process vessel, and when liquid charge is present in the process chamber at or above a predetermined level, the gaseous products in each headspace are isolated from each other, at least in part by the liquid charge.

2. A process vessel according to claim 1 in which the closure portions are non-floating.

3. A process vessel according to claim 1 in which at least one closure portion is removable from the process vessel separately to other closure portions to permit access to the interior of the receptacle.

4. A process vessel according to claim 1 in which each closure portion is configured to receive, in use, a portion of the liquid charge so that the gaseous products in each headspace are isolated from each other by the liquid charge present in each closure portion.

5. A process vessel according to claim 1 in the form of an anaerobic digester.

6. A process vessel according to claim 1 including three or more closure portions.

7. A process vessel according to claim 1 in which the closure portions consist essentially of a polymeric material or a composite material.

8. A process vessel according to claim 7 in which the closure portions consist essentially of glass-reinforced plastic (GRP) or a polyolefin.

9. A process vessel according to claim 1 in which at least one of the closure portions is tied to the receptacle by a tie arrangement which prevents or limits movement of the closure portion with respect to the receptacle.

10. A process vessel according to claim 1 in which the receptacle includes an internal flange portion which receives at least some of the closure portions.

11. A process vessel according to claim 10 in which at least some of the closure portions include a rim which is received by said internal flange portion.

12. A process vessel according to claim 11 in which at least some of the closure portions include a rim which is received by a rim of an adjacent closure portion so that the closure portions extend between opposite walls of the receptacle without any additional elements therebetween.

13. A process vessel according to claim 1 in which each of the closure portions has an associated gas outlet pipe which extends through a wall of the receptacle.

14. A process vessel according to claim 1 including the plurality of closure portions in combination with a fixed roof structure.

15. A process vessel according to claim 1 further including one or more walls disposed on to the floor of the receptacle.

16. A process vessel according to claim 15 in which the wall or walls are disposed in areas in which grit or sand is expected to accumulate, and one or more closure portions are located over said areas in which grit or sand is expected to accumulate.

17. A kit of parts for assembling a process vessel according claim 1.

18. A method of using a process vessel in a process in which a liquid charge produces a gaseous product, the method including the steps of:
providing a process vessel which includes a receptacle having an open upper end, and a plurality of closure portions for closing said open upper end, in which the process vessel is configured so that the liquid charge extends continuously within the process vessel; each closure portion is configured to provide a headspace in which the gaseous product can collect, and at least one closure portion is operable separately to other closure portions to permit access to the interior of the receptacle;
filling the process vessel with liquid charge to a level; and
collecting the gaseous product in the headspaces provided by the closure portion;
wherein liquid charge is present in the process chamber at or above a predetermined level so that the liquid charge, at least in part, isolates the gaseous products in each headspace from each other.

19. A method according to claim 18 in which the process vessel is filled so that each closure portion receives liquid charge, and the gaseous products in each headspace are isolated from each other by the liquid charge present in each closure portion.

20. A method according to claim 18 including the further step of operating one or more of the closure portions separately to other closure portions to permit access to the interior of the receptacle.
